(19) United States
(12) Patent Application Publication
(10) Pub. No.: US 2004/0259233 A1
(43) Pub. Date: Dec. 23, 2004

(54) Optimized device for the regulation and measurement of gas content in composting platforms or platforms for the treatment of waste with measuring probes

(76) Inventor: Philippe Thurot, Saintry (FR)

Correspondence Address: DAVID A. GUERRA, 933 OLANDER WAY SOUTH, SUITE 3, SOUTH PASADENA, FL, 33707, US

(21) Appl. No.: 10/501,158

(22) PCT Filed: Jan. 8, 2002
(86) PCT No.: PCT/FR02/00039

(51) Int. Cl. C12M 1/36; C05F 17/02

(52) U.S. Cl. 435/286.6; 435/290.1

(57) ABSTRACT

The invention relates to an optimized device for the regulation and measurement of the oxygen content or content of any other gas in composting platforms or platforms for the treatment of waste. The device comprises at least one offset cabinet (4) comprising one or several gas measurement probes (5), and in particular, at least one probe for the measurement of oxygen or CO₂; a gas suction pump (6), a programmable controller (9) which can comprise electrovalves which are integrated inside said programmable controller; an air supply pipe (11) which links each of the electrovalves (8) to a tube for the collection of gas (10) which can be placed in a pile or piles of waste or compost (14). According to the invention, the collection tube corresponds to one single electrovalve supply pipe, comprising an air suction basket (15); the electrovalves which are coupled to the pump succesively enable the suction of air and gases contained in the air for each tube and ensure that the air and gases are transmitted to a measuring probe or probes (5).

This invention is an optimized system for the regulation and discontinuous measurement of the oxygen content or the content of any other gas in composting or waste-processing platforms.

The invention involves more particularly a system for the discontinuous measurement of the gas content, and especially the oxygen content, of swath composed of compost, vegetation waste, fermentable waste or any other waste whose gas content must be measured.

Within the framework of this invention, the term “waste” designates all these types of waste indifferently.

The measuring system is designed to determine the gas content at a waste-processing platform and, in particular, at composting platforms or decontamination platforms for contaminated soil; a swath forming a pile of different kinds of waste or single pile covering a large surface area from several hundred to several thousand square meters.

The waste may be, in particular, ligneous, fibrous, muddy or earthy in nature, or even all or any of this.

Platforms designed for waste processing, and especially for composting or contaminated soil treatment, use systematically one or more measuring probes for determining gas content and, in particular, oxygen or C0₂ content with the sensor directly built into each probe. The probe thus has a sensor which sends the measurement signal directly to the programming system by means of a transmitter.

Numerous disadvantages arise in these measurement and regulation systems as the platforms increase in size and it becomes necessary to make gas, and in particular, oxygen measurements at several swath locations or at several points in the pile of waste.

Sensors are delicate instruments and there is increased risk that this equipment breaks down on the site.

There is not just one sensor but several measurement sensors used for measuring the amount of gas in the pile of waste.

Measurement probes are costly as each of them contains a sensor, signal measurement circuitry and a transmitter for sending the signal to the regulation system.

The integration of these various devices into the probe requires expertise in sealing techniques on the site in order to properly protect the electronic section of the sensors against humidity and dust.

The signal transmitted is electrical in nature which requires the measurement probe to be connected to the regulation and programming system by an electric wire.

In the case of composting or soil-decontamination platforms, the use of waste-overturning equipment may result in electric wires being torn out or measurement probes and their sensors being broken.

The use of probes, and especially oxygen probes, at the site requires regular and systematic calibration of all probes.

The probe(s) must be supplied electrical power in order to operate.

These drawbacks and these problems lead to high maintenance/servicing, probe calibration and cleaning costs when the probes have to be moved or the process has to be stopped when electric wires between the probes and the regulation system are torn out.

The purpose of the invention is to provide a measurement and regulation system that alleviates these drawbacks.

The objective of the invention is reached by means of a gas measurement and regulation system, and in particular the measurement of oxygen or CO₂, operating discontinuously. This system is comprised of a remote bay connected to the main regulation bay, containing a regulation and program controller, electric regulation valves integrated into, or not integrated into, the program controller; one or more gas-sampling rods, in particular for oxygen or CO₂ content; for measuring the ratio of oxygen in the pile of waste, in particular, an oxygen probe of the heated zirconium oxide sensor type; a single gas intake pump, in particular for oxygen; one or more rods for capturing, far down, the gases to be analyzed present in the air contained in the pile of waste, and in particular, the oxygen content in the pile or piles of waste.

The heated zirconium oxide sensors have a very short response time, on the order of a few seconds (less than ten seconds), and the time between recalibrations is about six months under normal operating conditions.

In accordance with the invention, the system also contains one or more rods for sampling gas, oxygen in particular. Each rod has a strainer inside of which emerges a plastic pipe that passes completely through the rod.

This pipe allows the air and gas in the pile of waste to be drawn in at this point. The plastic tube in each rod connects each rod to the electric regulation valves built into the remote bay. The rods are located away from the remote bay.

The air contained in the pile of waste is drawn in by means of the pump contained in the remote bay. The gas produced by the waste and contained in the air is consequently also drawn in up to the remote bay where the gas content is analyzed (zirconium probe for oxygen). A variation in the parameters related to the gas triggers, in relation to the specific parameters associated with the process, a regulation action for controlling and maintaining the specific parameters for platform operation, within given values (maintaining a concentration of oxygen between two boundary values for a composting platform operating system using controlled aeration functioning with aeration tunnels).

The system can be used, at the same time, to measure the amount of one or more gases contained in the air and characterizing this pile or all the swath forming the pile or the piles of waste.

The advantages of this system are many:

There are no more gas measurement sensors on the piles of waste and in the rods (no more gas measurement probes on the site, with sensors, and in particular in waste-overturning areas).

Only one probe for measuring the concentration of gas in the air for a given gas, such as oxygen or CO₂, and completely protected in the remote bay, supplying analyses of the concentration of this gas, for an entire platform, and successively for each swath whose physicochemical characteristics are to be examined.

The probes are eliminated. Henceforth, gas is sampled only through hollow rods driven into the pile(s) of waste.

There are no more electric wires between the rod and the regulation system, only a simple plastic pipe conducting air or the gases being analyzed in the remote bay.

Many rods can be installed as the cost of this equipment is very limited with respect to the cost of installing, in each rod, a measurement sensor and, its transmitter.

With a suitable controller having integrated inputs and outputs of the PT 100 and PT 1000 type, it is now possible to also eliminate the transmitter usually fitted in temperature probes, for operating composting and decontamination platforms.

For the system to operate properly, a certain number of conditions must be advantageously met:

A strainer is absolutely required at the ends of the rods driven into the waste otherwise the plastic air supply pipe may get clogged up.

Effective sealing is required from the strainer to the electric valves built into the regulation bay or directly coupled to the program controller; likewise, none of the plastic pipes can be pierced otherwise the air drawn in through each rod will not be the air that is to be analyzed inside the pile of waste or each pile of waste or a swath.

The pump installed in the remote bay must have enough power to draw in very quickly the air that is taken in successively through each rod.

The sensors for measuring the gas in the air sampled must be sensitive enough to provide a reliable and almost immediate measurement of the gas content being measured.

The gas analyzer should not take into account the gas content immediately drawn in and which was stagnating in the plastic pipe when pumping started as this data is false and does not correspond to the value to be measured. The only value to be measured is that obtained and which corresponds to the air actually drawn in from outside the rod and which did not stagnate in the plastic pipe.

The rod must be long enough to measure the gas content at the center of the waste pile.

The program controller that opens and closes the electric valves allowing the air and gases sampled successively in each rod to be drawn in should have enough capacity to control the opening and closing of the valves, the length of time these valves are opened and closed, and the time required for reading the relevant signal; reading the signals supplied by the gas probe or probes corresponding to other gases (oxygen probe or CO₂ probe) and the other measurement probes (temperature probes).

If it is desired to eliminate the transmitters operating in conjunction with the temperature probes, the program controller in the remote bay must incorporate PT 100 or PT 1000 analog input boards.

To protect the following equipment (probe or probes with measurement sensors, intake pump, regulation and program controller, and electric regulation valves), it is necessary to place all this equipment in one or more remote bays. This or these remote bays can be separate from the main bay which can collect all the technical data supplied by the measurement sensor or sensors.

At the upper part of the hollow rod, a packing gland or a simple coupling is sufficient enough for providing effective sealing between the rod and the plastic air and gas intake pipe as well as holding the latter in place.

The following description refers to FIGS. 1 to 4 which show a system for measuring the amount of gas in the air inside a pile of waste and corresponding to two preferred ways of implementing the invention.

The installation is comprised of a main control bay (1) containing the control computer which can be coupled to a control station or a computer (2). It also has, connected by a bus (3) to this main bay (1), a remote bay (4) containing one or more measurement probes (5) (one probe per type of gas being analyzed); this remote bay also contains a gas intake pump (6) and a mechanism for regulating and adjusting the flow of air to the pump (7); it houses several electric valves (8) operated by the program controller (9) (one electric valve per sampling rod (10)). Preferably, the gas intake pump (6) is equipped with a device for regulating the flow of air to the probe(s), of the rotameter type, for example.

The intake pump (6) draws in successively the air contained in each of the rods (10) and necessarily passing through the plastic pipes (11) connecting the gas sampling rods (10) to the electric valves (8).

The intake pump (6) is connected directly to the probe (5) (oxygen probe), otherwise to the other gas measurement probes through an air-supply pipe (11).

The gas probe(s) (5) are connected directly to the program controller (9) by means of an electric cable (12). The cable transmits the measurements obtained by each of the probes (5) to the program controller (9).

The program controller (9) is connected directly to the electric valves with electric cables (13) supplying the electrical signal giving the order to open or close the electric valves (8).

Gas is sampled in the waste pile(s) (14) by means of a rod through which a plastic pipe (11) passes.

Each plastic pipe (11) connects a sampling rod (10) to an electric valve (8).

A rod (10) has a strainer (15) at one of its ends and possibly a packing gland or coupling (16) at each end of the tube (17) (forming the rod (10), securing the plastic pipe in the body of the rod.

In accordance with one system implementation approach shown in FIG. 2, the programming unit can have input and output boards incorporating the electric valves directly and PT 100 and PT 1000-type input and output boards (temperature) allowing the transmitters usually fitted in each temperature probe to be eliminated.

It is obvious that the number of electric valves depends on the size of the platform and the number of measurements desired for analysis.

It is obvious that the remote bay can contain a single measurement probe for only one type of gas in the same way that it can contain several probes for analyzing, with the same system, several types of gas present in the waste pile(s).

In accordance with a variant of the implementation approaches shown, the analysis equipment normally integrated into a single remote bay can be integrated into one, two, or several remote bays depending on operating and process constraints.

The system presented has the following numerous advantages:

elimination of the gas measurement sensors on the waste piles and integrated into

the rods; simplicity, reliability, and economy for the system;

easy replacement of the plastic pipes in the event that these are torn out by machinery operating on the platforms;

a reduction in the number of sensors and, in particular, sensors used for measuring oxygen or CO₂ content for the operation of composting or waste-processing platforms (a single sensor or else only two or three sensors may suffice for operating composting platforms for several tens of thousands of tons).

the possibility of having a single oxygen sensor for operating and regulating a composting platform processing several thousands of tons of vegetation waste, mud, and fermentable waste, or contaminated soil.

the possible elimination of the temperature signal transmitter at each temperature probe.

the possibility of having a single standard model rod able to accommodate either the plastic air-supply pipe or a PT 100 or PT 1000, type probe, or any other type of sensor (humidity sensor).

the oxygen analysis probe for a composting platform may no longer drift and consequently require recalibration that is less frequent than previously.

in this system, if recalibration must nonetheless be carried out, it might involve one, or maybe two or three, oxygen probes only.

The invention finds a whole series of privileged applications in the area of the composting of all types of organic waste, and especially vegetation waste, mud, and fermentable waste, processed separately or mixed together.

It also finds privileged applications in the area of the treatment of contaminated soil.

1. An optimized system for the regulation and discontinuous measurement of the oxygen content or the content of any other gas in platforms for composting or processing waste, especially in the form of swaths, the system being comprised of at least one remote bay (4) itself containing one or more gas measurement probes (5) and, in particular, at least one oxygen or C0₂ measurement probes; a gas intake pump (6); electric valves (8) operated by a program controller (9); a pipe (11) connecting each of the electric valves (8) to a gas sampling device, the electric valves (8) coupled to the pump (6) allowing the air and the gases contained in this air at each sampling device (10) to be drawn in successively and sent to the measurement probe(s) (5), characterized by the fact that the sampling device is a rod (10) with two opposite ends able to be driven into the pile(s) of waste or compost; each one of the sampling rods (10) corresponding to one single pipe and carrying an air intake strainer (15) at one end, the pipe being connected at the other end of the rod and by the fact that the oxygen measurement probe (5) must be able to supply within a very short response time the measurement of the oxygen content of several swaths and that consequently this probe should be of the heated zirconium oxide sensor type with a very short response time, on the order of a few seconds and less than ten seconds.

2. A system as in claim 1, characterized by the fact that the electric valves (8) are separate from the program controller (9) or are integrated directly into the program controller (9).

3. A system as in one of the claims in 1 or 2, characterized by the fact that the attachment of the rod to the pipe uses a packing gland or a coupling facilitating the fastening or insertion of the pipe.

4. A system as in one of the claims in 1 to 3, characterized by the fact that a single gas measurement probe (5), in particular oxygen or CO₂, can be used to measure, respectively, the oxygen or C0₂ content of several swaths by means of samples obtained from the various gas sampling rods (10).

5. A system as in one of the claims in 1 to 4, characterized by the fact that the program controller (9) has temperature probes and one or more input/output ports enabling it to receive PT 100 or PT 1000-type signals for temperature measurement or other signals for measuring other gases present.

6. A system as in one of the claims in 1 to 5, characterized by the fact that the rod (10) for sampling air in the waste, and used for measuring the concentration of oxygen, CO₂, or any other gas, does not contain a sensor or a transmitter built into the body of the rod, but only a strainer (15) and an air-supply pipe (11).

7. A system as in one of the claims in 1 to 6, characterized by the fact that the gas intake pump (6) can be equipped with a device (7) for regulating the flow of air to the probe(s), of the rotameter type.

8. A system as in one of the claims in 1 to 7, characterized by the fact that the air-sampling rod (10) contains, at one of its ends, an air strainer (15) and, at the other end, a packing gland or a coupling enabling insertion of the pipe (11) for sampling the air in the compost or waste.
